SEQUENCE LISTING (6 sequences)

SEQ ID NO: 1 - protein, 57 aa, Homo sapiens
MKFFVFALVLALMISMISADSHEKRHHGYRRKFHEKHHSHREFPFYGDYGSNYLYDN

SEQ ID NO: 2 - protein, 38 aa, Homo sapiens
DSHEKRHHGYRRKFHEKHHSHREFPFYGDYGSNYLYDN

SEQ ID NO: 3 - DNA, 23 bp, Homo sapiens
cgctgattcacatgaaaagagac

SEQ ID NO: 4 - DNA, 23 bp, Homo sapiens
agggaagtatcatgaaacacaga

SEQ ID NO: 5 - RNA, 25 nt, Homo sapiens
uauagauaauuugauccauaguccc

SEQ ID NO: 6 - RNA, 25 nt, Homo sapiens
aaaucaugagagccaagacuaaagc

(19) United States
(12) Patent Application Publication
(10) Pub. No.: US 2013/0273653 A1
(43) Pub. Date: Oct. 17, 2013

(54) DIAGNOSTIC MARKER FOR MELANOMA, PHARMACEUTICAL COMPOSITION COMPRISING INHIBITOR OF THE FUNCTION OF THE HISTATIN-1 PROTEIN AND IMMUNOSUPPRESSOR

(71) Applicants: Tomonori Yaguchi, Shinjuku-ku (JP); Yutaka Kawakami, Shinjuku-ku (JP)

(72) Inventors: Tomonori Yaguchi, Shinjuku-ku (JP); Yutaka Kawakami, Shinjuku-ku (JP)

(73) Assignee: KEIO UNIVERSITY, Minato-ku (JP)

(21) Appl. No.: 13/817,336

(22) PCT Filed: Aug. 18, 2011
(86) PCT No.: PCT/JP11/68702

§ 371 Date: Jun. 11, 2013

(30) Foreign Application Priority Data
Aug. 19, 2010 (JP) 2010-184150

(51) Int. Cl. C07K 14/47

(52) U.S. Cl. CPC C07K 14/47
USPC 435/372; 435/320.1; 530/350; 435/377

(57) ABSTRACT

The present invention is directed to provide diagnostic markers for melanoma, novel pharmaceutical compositions, and novel immunosuppressors. The histatin-1 mRNA and the histatin-1 protein can be used as the diagnostic markers for melanoma. In addition an inhibitor of the function that inhibits the function of the histatin-1 protein may be used as a pharmaceutical composition. Furthermore, a dendritic cell differentiated from a monocyte by the action of the histatin-1 protein or the histatin-1 protein may be used as an immunosuppressor.

### CROSS REFERENCE TO RELATED APPLICATION

This application claims the benefit of Japanese Patent Application No. 2010-184150 filed on Aug. 19, 2010, the entire disclosure of which is hereby incorporated by reference.

### TECHNICAL FIELD

The present invention relates to diagnostic markers for melanoma, pharmaceutical compositions comprising an inhibitor of the function of the histatin-1 protein, and immunosuppressors.

### BACKGROUND ART

Melanoma is a malignant skin tumor caused by malignant transformation of melanocytes or nevus cells. It is a tumor of high malignancy with particularly high incidence among fair-skinned people. However, since patients with melanoma in stage I or II have a good prognosis, early detection of melanoma through appropriate diagnostic testing is important. Current practice for the diagnosis of melanoma and the determination of stages relies on biopsy of tumor tissues.

Histatin-1 protein is known to be contained in saliva (see, for example, Vitorino R. et al., Biomed. Chromatogr. 19 (3): 214-222, 2005) and is also known to have an antibacterial action (see, for example, Japanese Patent Laid-open No. 2005-154338). No findings, however, have been reported which show a relationship between the histatin-1 gene or the histatin-1 protein and a cancer.

In addition, no findings have been reported,which show any relationship between the histatin-1 gene or the histatin-1 protein and a mechanism of immunosuppression.

### Aim of the Invention

The present invention is directed to provide diagnostic markers for melanoma, novel pharmaceutical compositions, and novel immunosuppressors.

### SUMMARY OF THE INVENTION

The present inventors have found that histatin-1 mRNA and histatin-1 protein are detected in organism-derived samples obtained from melanoma patients and thus invented biomarkers for diagnosing melanoma.

More specifically, a biomarker according to the present invention is for use in diagnosing melanoma, the biomarker being histatin-1 mRNA or histatin-1 protein.

A measurement method according to the present invention comprises the step of measuring the content of histatin-1 mRNA or the content of histatin-1 protein in a collected organism-derived sample. The organism-derived sample is preferably skin or blood.

A method of determining the efficacy of a drug on melanoma according to the present invention comprises the steps of (1) measuring the amount of histatin-1 mRNA or histatin-1 protein contained in organism-derived samples obtained from a vertebrate having melanoma before and after the administration of a drug to the vertebrate, the vertebrate being human or a non-human animal, and (2) comparing the measurements obtained in the step 1 before and after the administration of the drug.

A method of determining the efficacy of a drug on melanoma according to the present invention comprises the steps of (1) measuring the amount of histatin-1 mRNA or histatin-1 protein contained in an organism-derived sample obtained from a vertebrate having melanoma after the administration of the drug to the vertebrate, the vertebrate being human or a non-human animal, and (2) comparing the measurement obtained in the step 1 with a threshold for the content of the histatin-1 mRNA or the histatin-1 protein, the threshold being used to discriminate vertebrates having melanoma and normal vertebrates.

A method of screening a therapeutic agent for human or a non-human vertebrate having melanoma according to the present invention comprises the steps of (1) obtaining an organism-derived sample from an animal model of melanoma before the administration of a candidate substance for a therapeutic agent to the model, (2) administering the candidate substance for a therapeutic agent to the animal model of melanoma, (3) collecting an organism-derived sample from the animal model of melanoma after the administration of the candidate substance, (4) measuring the content of histatin-1 mRNA or histatin-1 protein in the organism-derived samples collected in the steps 1 and 3, and (5) comparing the measurements in the step 4 between for the case of before the administration of the candidate substance for the therapeutic agent and for the case of after the administration of the substance.

In addition, the present inventors have found a relationship between histatin-1 gene as well as histatin-1 protein and migration capacity of melanoma. Thus, an invention of pharmaceutical compositions comprising an inhibitor of the function of the histatin-1 protein was completed.

More specifically, a pharmaceutical composition according to the present invention comprises an inhibitor of the function that inhibits the function of the histatin-1 protein. The inhibitor of the function is preferably an antibody specific for the histatin-1 protein or an inhibitor of the expression that inhibits the expression of the histatin-1 protein. The inhibitor of the expression is preferably siRNA.

A therapeutic agent for melanoma according to the present invention comprises any one of the aforementioned inhibitors of the function that inhibit the function of the histatin-1 protein.

Furthermore, the present inventors have found that monocytes can be differentiated into immunosuppressive dendritic cells when histatin-1 protein acts on the monocytes. An invention of the immunosuppressors according to the present invention was thus completed.

More specifically, an immunosuppressor according to the present invention comprises the histatin-1 protein. The immunosuppressor according to the present invention may contain an expression vector capable of expressing the histatin-1 protein. Alternatively, the immunosuppressor may contain a transformant transformed with an expression vector capable of expressing the histatin-1 protein.

A method of producing an immunosuppressive dendritic cell according to the present invention comprises the step of differentiating a monocyte isolated from human or a non-human vertebrate in the presence of histatin-1 protein.

An immunosuppressive dendritic cell according to the present invention is produced by a method of production comprising the step of differentiating a monocyte isolated from human or a non-human vertebrate in the presence of histatin-1 protein. The immunosuppressor according to the present invention may comprise the immunosuppressive dendritic cell.

### BRIEF DESCRIPTION OF THE DRAWINGS

FIG. 1 is a figure showing the expression of histatin-1 mRNA in human melanoma cell lines in an embodiment of the present invention;

FIG. 2 is a graph showing the survival rate of melanoma patients in the groups with a high or low expression of histatin-1 mRNA in metastatic lesions in their sentinel lymph nodes in an embodiment of the present invention;

FIG. 3 is a graph showing an increase in migration capacity of cells in the case that histatin-1 protein was added to a human melanoma cell line C32mel in an embodiment of the present invention;

FIG. 4 is a graph showing a decrease in migration capacity of cells in the case that histatin-1 gene was knocked down in a human melanoma cell line SKmel23 in an embodiment of the present invention;

FIG. 5 is a graph showing an increase in migration capacity of cells in the case that histatin-1 gene was forcibly expressed in a human melanoma cell line A375 in an embodiment of the present invention;

FIG. 6 shows graphical representations of (A) FACS analyses showing invasion and metastasis of implanted cells into the associated lymph nodes and (B) distribution of results obtained from individuals in the MOCK and histatin-1 groups, in the cases that no tumor (NO TUMOR), tumor cells not expressing histatin-1 (MOCK) or tumor cells expressing histatin-1 (HISTAIN-1) were implanted into mice, in an embodiment of the present invention;

FIG. 7 shows graphical representations of (A) FACS analyses showing invasion and metastasis of implanted cells into lung tissues and (B) distribution of results obtained from individuals in the MOCK and histatin-1 groups, in the cases that no tumor (NO TUMOR), tumor cells not expressing histatin-1 (MOCK) or tumor cells expressing histatin-1 (HISTAIN-1) were implanted into mice, in an embodiment of the present invention;

FIG. 8 shows graphical representations of the amount of (A) IL-12, (B) TNF-α and (C) IL-10, which were produced by dendritic differentiated from monocytes by the action of histatin-1 protein in an embodiment of the present invention;

FIG. 9 show graphs of expression of cell surface markers in dendritic cells differentiated from monocytes by the action of histatin-1 protein in an embodiment of the present invention;

FIG. 10 shows a graph of the amount of IFN-γ produced by T cells when the T cells were incubated along with dendritic cells differentiated from monocytes by the action of histatin-1 protein in an embodiment of the present invention; and

FIG. 11 shows graphs of the amount of IFN-γ produced by T cells when 397mel in which histatin-1 protein was forcibly expressed was implanted into nude mice and dendritic cells isolated from tumor tissue derived from the 397mel or spleen were incubated along with the T cells. 397mel in which histatin-1 protein was not forcibly expressed was used as a negative control.

### EMBODIMENTS FOR CARRYING OUT THE INVENTION

Embodiments of the present invention that were completed based on the aforementioned findings are described in detail in reference to Examples.

Unless otherwise noted in embodiments and examples, all procedures used are as described in standard protocols such as J. Sambrook, E. F. Fritsch & T. Maniatis (Ed.), Molecular cloning, a laboratory manual (3rd edition), Cold Spring Harbor Press, Cold Spring Harbor, New York (2001); F. M. Ausubel, R. Brent, R. E. Kingston, D. D. Moore, J. G. Seidman, J. A. Smith, K. Struhl (Ed.), Current Protocols in Molecular Biology, John Wiley & Sons Ltd., with or without modifications or changes. In addition, unless otherwise noted, a commercial reagent kit or a measurement instrument, if any, is used as described according to protocols attached thereto.

The objects, features, advantages, and ideas of the present invention are apparent to those skilled in the art from consideration of the description of this specification. Furthermore, those skilled in the art can easily reproduce the present invention from these descriptions. The embodiments and specific examples described below represent preferable ones of the present invention, which are given for the purpose of illustration or explanation. The present invention is not limited thereto. It is obvious to those skilled in the art that various modifications may be made according to the descriptions of the present specification without departing from the intension and scope of the present invention disclosed herein.

As used herein, the term melanoma refers to all types of melanoma including lentigo maligna melanoma, superficial spreading melanoma, nodular melanoma, acral lentiginous melanoma, and melanoma with a mixed character. The term is not limited by the stage of melanoma.

### ==Biomarkers==

As used herein, biomarkers for diagnosing melanoma (diagnostic markers) include, for example, diagnostic markers with which animals having melanoma can be diagnosed with a high probability (disease markers), diagnostic markers with which the stage of melanoma can be determined (stage markers), diagnostic markers with which malignancy of melanoma can be determined (malignancy markers), diagnostic markers for determination of the prognosis or the life expectancy of animals having melanoma (prognosis markers), and diagnostic markers with which invasive potential or metastatic potential of melanoma can be determined (potential markers of invasion or potential markers of metastasis).

The biomarkers according to the present invention are used as an indicator of the expression level of histatin-1 gene. Histatin-1 mRNA and histatin-1 protein serve as the biomarkers. The biomarkers can be used to diagnose melanoma in a vertebrate to be diagnosed by measuring the content of the biomarker in an organism-derived sample obtained from a vertebrate to be diagnosed as well as to make judgment of or make screening for drugs for melanoma.

The animal to be diagnosed may be human or a non-human vertebrate as long as it has a biomarker according to the present invention. The animal is, however, preferably mammal such as human, a mouse, a rat, a dog, a cat, a horse, sheep, a rabbit, a pig, and a monkey. It is most preferable that the animal is human. No specific limitation is placed upon the age and sex of the vertebrate.

The content of biomarkers (hereinafter, also referred to as the amount of biomarkers) is preferably expressed as an absolute concentration of the biomarkers. No specific limitation is, however, placed on what measure is used to describe the content of the biomarkers as long as the value is proportional to the absolute concentration and the absolute concentrations of the biomarker in the individuals can be compared by the value; thus, the content may be expressed as a relative concentration, the content per a unit volume or raw data measured to determine the absolute concentration.

### ==Method of Measuring Biomarkers==

In the present invention, melanoma is diagnosed by measuring an expression level of the histatin-1 gene in an organism-derived sample obtained from an animal to be diagnosed. The type of the organism-derived sample used to measure the expression level of the histatin-1 gene is not specifically limited. It is, however, preferable that the organism-derived sample is a skin tissue suspected to be melanoma in the animal to be diagnosed, or blood collected from the animal to be diagnosed, considering that they are easily obtainable. The expression level of the histatin-1 gene may be determined by measuring, for example, the content of the histatin-1 mRNA or the histatin-1 protein using a method well-known to those skilled in the art.

[Histatin-1 mRNAs]

Histatin-1 mRNA can be measured by detecting it in the organism-derived sample obtained from the animal to be diagnosed with, for example, in situ hybridization using probes specific for the histatin-1 mRNA. The content of the histatin-1 mRNA in the organism-derived sample to be diagnosed can be calculated by comparing a predetermined standard curve showing the content of the histatin-1 mRNA with respect to the amount of the labels in the organism-derived samples with the amount of the labels in the organism-derived sample to be diagnosed. Alternatively, according to a method well-known to those skilled in the art, cDNA may be prerpared from mRNA isolated from the organism-derived sample and the content of the histatin-1 mRNA may be determined by PCR using a pair of primers specific for the histatin-1. Alternatively, RNA is isolated from the organism-derived sample and the histatin-1 mRNA may be detected through Northern Blotting using probes specific for the histatin-1 mRNA. By comparing the detected histatin-1 mRNA with a predetermined standard curve, its content in the organism-derived sample can be calculated.

### [Histatin-1 Protein]

The content of the histatin-1 protein may be measured by using, for example, an antibody specific for the histatin-1 protein in the organism-derived sample obtained from the animal to be diagnosed. The antibody specific for the histatin-1 protein may be polyclonal or monoclonal, and is not limited by the animal species from which the antibody is derived. The antibody comprises full-length immunoglobulin and partial antibody. The partial antibody refers to a fragment of the antibody with the antigen-binding site having an antigen-binding activity. Examples of the partial antibody include a Fab fragment and a F(ab′), fragment.

When the content of the histatin-1 protein is measured in the organism-derived sample such as skin tissue, the histatin-1 protein may be detected using, for example, an immunohistochemical technique. Alternatively, using a method well-known to those skilled in the art, proteins are extracted from the tissue of the animal to be diagnosed and the histatin-1 protein may be detected through Western Blotting. The content of the histatin-1 protein thus detected in the tissue can be calculated from a predetermined standard curve. On the other hand, when blood is used as the organism-derived sample, the content of the histatin-1 protein can be measured using a well-known method. For example, a well-known method can be used such as ELISA (enzyme-linked immunosorbent assay) including direct competitive ELISA, indirect competitive ELISA, and sandwich ELISA, RIA (radioimmunoassay), flowmetry, and immunochromatography. It is preferable that the blood to be subject to the measurement is pretreated. For example, it is preferable that serum or plasma is separated from the blood through standing or centrifugation and the serum or plasma collected is used for the measurement.

The probe or antibody used in this method to detect the biomarker is preferably labeled for the purpose of visualization. Examples of the labeling substance include, but not limited to, fluorescent substances (e.g., FITC, rhodamine, and phalloidine), colloidal particles such as gold, fluorescent microbeads such as Luminex (registered trademark, Luminex Corporation), heavy metals (e.g., gold and platinum), chromoproteins (e.g., phycoerythrin and phycocyanin), radioisotopes (e.g., ¹H, ¹⁴C, ³²P, ³⁵S, ¹²⁵I and ¹³¹I), enzymes (e.g., peroxidase and alkaline phosphatase), biotin and streptavidin.

### ==Method of Using the Biomarkers==

The biomarkers according to the present invention can be used for the diagnosis of melanoma, including the diagnosis of melanoma or the determination of a stage of melanoma.

### [Diagnosis of Melanoma]

For example, a range of the amount of the biomarkers or a threshold for use in diagnosis has been determined in advance in order to determine whether a given animal has melanoma using the biomarker according to, the present invention.

More specifically, the amount of the biomarker is measured in the organism-derived sample obtained from vertebrates that have been diagnosed to have melanoma (also referred herein to affected animal) and vertebrates that have been diagnosed to have no melanoma (also referred herein to normal animals) and their distributions are determined. When the amount of the biomarker in the organism-derived sample obtained from the vertebrate to be diagnosed falls within the range of the amount of the biomarker associated with the affected animals, the subject vertebrate is determined to have melanoma. On the other hand, when the amount in question falls within the range of the amount of the biomarker associated with the normal animals, the subject vertebrate is determined to have no melanoma. Alternatively, only the range of the amount of the biomarker associated with the normal animals may be used for the diagnosis. In this case, the vertebrate with a value falling within the range may be determined to be normal and otherwise be determined to have melanoma.

Alternatively, in place of a range of the amount of the biomarker in the organism-derived samples of the normal animals, the amount of the biomarker in the organism-derived samples obtained when the vertebrate to be diagnosed is in a normal state may be measured in advance, and a range of the amount of the biomarker in the normal state may be determined. Then the amount of the biomarker upon diagnosis can be compared with the amount of the biomarker in the normal state. If the amount of the biomarkers upon diagnosis falls within the range of the amount of the biomarkers in the normal state, the vertebrate is determined to have no melanoma.

The range of the amount of the biomarker in the organism-derived samples of the affected animal or the normal animal may be set as the same range of a span of the actual contents, as described above. The range is, however, not specifically limited. It may be defined as a range between a value obtained by subtracting the standard deviation from the average of the measured values for the biomarker in the organism-derived samples obtained from two or more vertebrates and a value obtained by adding the standard deviation to the same average. Alternatively, the range may be defined as a range between the lower limit and the upper limit of the averages. In addition, the threshold used to discriminate between an affected animal and a normal animal may be determined according to any one of ordinary methods known to those skilled in the art. For example, melanoma can be diagnosed in the animal to be diagnosed more precisely by determining a threshold in such a manner that as many affected animals as possible are included in a range of values equal to or greater than the threshold and as many normal animals as possible are included in a range of values smaller than the threshold.

The diagnosis of melanoma using a biomarker according to the present invention may be combined with a conventional procedure such as visual inspection or biopsy.

### [Determination of Stage of Melanoma]

The progression of melanoma is typically categorized into four stages, i.e., stages I-IV. Stage I is the most early and stage IV is the most advanced. A biomarker according to the present invention may be used for determining the stage (degree of progression) of melanoma.

More specifically, the amount of the biomarker has been measured in advance in the organism-derived samples obtained from normal animals and vertebrates having melanoma in stage I, II, III, or IV. As in the case of the aforementioned “Diagnosis of melanoma”, ranges of the amount of the biomarker corresponding to the respective stages of melanoma or thresholds that can be used to distinguish the stages are determined. The degree of progression of melanoma may be determined according to which range the amount of the biomarkers in the vertebrate to be diagnosed falls.

### [Prediction of Prognosis After Treatment of Melanoma]

It is known that prognosis of an affected animal having melanoma tends to be good when the disease is in an early stage while the prognosis tends to be bad when the melanoma is in an advanced stage. For example, it is said that the percentage of patients that are alive five years or longer after the initiation of treatment (five-year survival rate) is 95 to 100%, 70 to 80%, 50 to 60%, and around 10% for the stages I, II, III, and IV, respectively. Thus, the biomarkers according to the present invention may be used for predicting the prognosis after treatment of melanoma. More specifically, the larger the amount of a biomarker upon diagnosis s, the poorer the prognosis i The smaller the amount is, the better the prognosis is Alternatively, the prognosis may be predicted to be poor when the amount of the biomarker upon diagnosis is larger than a predetermined value, and is predicted to be good when the amount is smaller than the predetermined value.

The prediction of the prognosis according to the present invention may be done before, during, or after treatment of melanoma. An prognosis expected at each of the point can be predicted.

The term good prognosis indicates that the likely outcome after treatment of melanoma is good. The term poor prognosis indicates that the likely outcome after treatment of melanoma is not good. For example, as an indicator of the prognosis, ranges or thresholds of the amount of the biomarker may be determined in advance in association with survival years after the initiation of treatment. Alternatively, the five-year survival rate that is typically used for predicting the prognosis of cancer patients may be used to determine, in advance, a threshold or a range of the amount of the biomarkers corresponding to good or poor case in which patients expected to be alive five years or longer after the initiation of the treatment is considered to have good prognosis, and those expected to die within five years is considered to have poor prognosis.

### [Determination of Invasive Potential and Metastatic Potential of Melanoma]

The biomarkers according to the present invention may be used to determine invasive potential, metastatic potential, and malignancy of melanoma.

More specifically, the amount of the biomarker of organism-derived samples obtained from normal animals and vertebrates having melanoma with different invasive potentials or metastatic potentials is measured in advance. As in the case of the aforementioned “Diagnosis of melanoma”, determined are a range of the amount of the biomarkers corresponding to the invasive potential or the metastatic potential of melanoma or a threshold with which the degree of the invasive potential or the metastatic potential can be determined. The invasive potential or the metastatic potential of melanoma can be determined depending on which one of ranges the amount of the biomarker in the vertebrate to be diagnosed falls in. More specifically, the greater the amount of the biomarker upon diagnosis is, the higher the invasive potential or the metastatic potential is. The smaller the amount is, the lower the invasive potential or the metastatic potential is. Alternatively, the invasive potential or the metastatic potential may be considered to be high and low when the amount of the biomarkers upon diagnosis is larger or smaller, respectively, than a predetermined value.

### [Determination of Malignancy of Melanoma]

For tumors, melanoma is considered to have relatively high malignancy when the tumor cells proliferate fast, the tumor cells have a high invasive potential, the tumor cells have a high metastatic potential and/or prognosis is poor. On the other hand, melanoma is considered to have relatively low malignancy when the tumor cells proliferate slowly, the tumor cells have a low invasive potential, the tumor cells have a low metastatic potential and/or prognosis is good. Thus, the biomarkers according to the present invention can be used to determine malignancy of melanoma. More specifically, the larger the amount of the biomarker upon diagnosis is, the higher the malignancy. The smaller the amount is, the lower the malignancy is. Alternatively, the malignancy may be determined to be high and low when the amount of the biomarkers upon diagnosis is larger and smaller, respectively, than a predetermined value.

### [Determination of Efficacy of Drug]

The biomarkers according to the present invention may be used for determining the efficacy of a drug for melanoma.

The efficacy of a therapeutic agent against a given disease may vary from person to person. Accordingly, it is significantly useful to determine the efficacy of a given therapeutic agent in a particular individual. With the biomarkers according to the present invention, the efficacy of a therapeutic agent against melanoma can be determined easily. For example, it is possible to determine whether melanoma is improved by diagnosing melanoma using the biomarker according to the present invention before and after the administration of a therapeutic agent for melanoma. Based on this, the efficacy of the therapeutic agent administered can be determined.

### [Screening of Drug Effective for Melanoma]

A compound effective in treating, melanoma can be identified using the biomarkers according to the present invention in an animal model of melanoma. For example, a candidate compound for developing a therapeutic agent for melanoma is administered to an animal model of melanoma. The organism-derived samples are collected before and after the administration. Melanoma is then diagnosed using the biomarker according to the present invention to determine whether the melanoma is alleviated. In this way, the use of the biomarkers according to the present invention allows easy screening of a compound effective in treating melanoma.

### [Screening for Drug Most Effective in an Affected Animal]

For drugs that vary in efficacy from individual to individual, it is possible to screen for the most effective drug for a given vertebrate with melanoma by adminstering two or more drugs separately to the vertebrate and then determining the efficacy of each drug using the biomarker.

### ==Pharmaceutical Compositions==

Pharmaceutical compositions according to the present invention comprise an inhibitor of the function that inhibits the function of the histatin-1 protein.

The inhibitor of the function of the histatin-1 protein is not limited in its mechanism as long as it inhibits the function of the histatin-1 protein in the whole cell where it otherwise should function in the animal to which the pharmaceutical composition is administered. The inhibitor of the function may be a substance that inhibits the function of the histatin-1 protein itself. Alternatively, the inhibitor of the function may be an expression inhibitor that inhibits the function of the histatin-1 protein by inhibiting its expression. The substance that inhibits the function of the histatin-1 protein itself may act by means of, for example, inhibiting somewhere in the mechanism of action of the histatin-1 protein in cells. Alternatively, the inhibitor of the function may be a substance that acts on the histatin-1 protein itself to inhibit the function of the histatin-1 protein. Examples of the substance that inhibits the mechanism of action of the histatin-1 protein in cells include, 1,4-diamino-2,3-dicyano-1,4-bis(2-aminophenylthio)butadiene (see, U0126, Oudhoff M J et al., FASEB J. 22: 3805 2008). Examples of the substance that acts on the histatin-1 protein itself include specific antibodies against the histatin-1 protein. The specific antibodies against the histatin-1 protein may be polyclonal or monoclonal, and is not limited by the animal species from which the antibody is derived. The antibody comprises full-length immunoglobulin and partial antibody. The partial antibody refers to a fragment of antibody with the antigen-binding site having antigen-binding activity. Examples of the partial antibody include a Fab fragment and a F(ab′)₂ fragment. The expression inhibitor is not specifically limited as long as it can inhibit the expression of the histatin-1 gene. Examples of the expression inhibitor include antisense nucleic acids against transcription products of the histatin-1 gene or a portion thereof, nucleic acids having ribozyme activity against transcription products of the histatin-1 gene as well as siRNA, shRNA, miRNA, aptamers, and decoys to the histatin-1 gene. Methods of using these nucleic acids are in the technical knowledge of those skilled in the art, and nucleic acids for that purpose can easily be designed by those skilled in the art. These nucleic acids may be chemically synthesized and used as a pharmaceutical composition without any further purification. For RNA, however, a suitable expression vector capable of expressing RNA may be formulated as the pharmaceutical composition and administered to the subject affected animal to express the RNA in the body of the animal.

Such pharmaceutical compositions may be formulated through a method well-known to those skilled in the art to prepare a therapeutic agent for melanoma. The therapeutic agent for melanoma is formulated using pharmaceutical additives well-known to those skilled in the art, such as a pharmaceutically-acceptable carrier, diluent and vehicle. The dosage form is not specifically limited as long as it is suitable for delivering the present medicament to a lesion within the body of the animal to which the medicament is to be administered. The medicament may be formulated into oral drugs such as a tablet, a capsule, granules, powders, syrup, a enteric-coated tablet, a controlled-release capsule, a cachou, a chewable tablet, a drop, a pill, oral liquid, a confectionery tablet, a controlled-release tablet, and controlled-release granules. The medicament may be formulated into injectables. This medicament may be combined with a different pharmaceutical composition as well as the aforementioned pharmaceutical additives.

The animal to which the therapeutic agent for melanoma thus prepared is to be administered is not specifically limited as long as it has the histatin-1 protein, but a vertebrate is preferable. The animal may be human or a non-vertebrate animal. The animal is, however, preferably mammal such as human, mouse, rat, dog, cat, horse, sheep, rabbit, pig, and monkey. It is most preferable that the animal is human. No specific limitation is placed upon the age and sex of the vertebrate.

The route of administration is not specifically limited and examples include intracutaneous, subcutaneous, intravenous, intralymphatic, and intraperitoneal administrations. The physician may select it appropriately.

### ==Immunosuppressors==

Immunosuppressors according to the present invention contain the histatin-1 protein, an expression vector capable of expressing the histatin-1 protein, a transformant transformed with an expression vector capable of expressing the histatin-1 protein or an immunosuppressive dendritic cell differentiated from a monocyte by the action of the histatin-1 protein.

The animal to which the immunosuppressor is to be administered is preferably a vertebrate. The animal may be human or a non-vertebrate animal. The animal is, however, preferably mammal such as human, mouse, rat, dog, cat, horse, sheep, rabbit, pig, and monkey. It is most preferable that the animal is human. No specific limitation is placed upon the age and sex of the vertebrate.

With regard to the immunosuppressors, when the histatin-1 protein is directly or indirectly administered, such as in the case of the histatin-1 protein, the expression vector or the transformant, the histatin-1 protein is preferably derived from the animal to which the immunosuppressor is to be administered. However, the immunosuppressor is not specifically limited as long as it acts on monocytes derived from the animal to which the immunosuppressor is to be administered and induces them to differentiate into immunosuppressive dendritic cells. Examples include, when the animal to which the immunosuppressor is to be administered is human, human histatin-1 protein (DSHEKRHHGYRRKFHEKHHSHREFPFYGDYGSNYLYDN; SEQ ID No. 2) in which the signal sequence is removed from the protein (MKFFVFALVLALMISMISADSHEKRHHGYRRKFHEKHHSHREFPFYGDYGSNYLYDN; SEQ IS No. 1) of GenBank No. M26664, well as homologues (including orthologues) of other animal species having an the differentiation of human monocytes into immunosuppressive dendritic cells. In addition, when the immunosuppressive dendritic cells are to be administered, the histatin-1 protein is preferably derived from the animal from which the immunosuppressive dendritic cells have been derived. The histatin-1 protein is, however, not specifically limited as long as it has an action of inducing monocytes derived from the subject animal to differentiate into immunosuppressive dendritic cells. When the animal is human, the histatin-1 protein may be not only the human histatin-1 protein but also histatin-1 homologues (including orthologs) of other animal species having an action of inducing the differentiation of human monocytes into immunosuppressive dendritic cells. The animal from which the immunosuppressive dendritic cells have been derived is preferably of the same species as the animal to which the immunosuppressor is to be administered. The animal may be, however, of a different species from the animal to which the immunosuppressor is to be administered as long as the intended purpose is not significantly affected. The physician may appropriately select the animal species.

The histatin-1 protein contained in the immunosuppressor may have an amino acid sequence with one or more alterations such as deletion, insertion, or substitution of several amino acid residues (e.g., 10 amino acid residues, preferably 8 amino acid residues, more preferably 6 amino acid residues, yet more preferably, 4 amino acid residues, and further preferably 2 amino acid residues) as long as it has a desired immunosuppressive function.

Examples of the diseases to which the immunosuppressors are applicable include autoimmune diseases such as rheumatoid arthritis and inflammatory bowel diseases, and graft-versus-host disease (GVHD) following a tissue transplant.

A method of obtaining the histatin-1 protein is not specifically limited. The peptides may be isolated and purified from the cells expressing each of them. Alternatively, they may be recombinant peptides produced by recombination or peptides that are chemically synthesized using a well-known technique. The histatin-1 protein may be modified not to subject to in vivo degradation. The expression vector capable of expressing the histatin-1 protein may be prepared by inserting DNA encoding the histatin-1 protein into an appropriate expression vector using a recombination technique well-known to those skilled in the art. On the other hand, transformants may be generated by introducing such an expression vector into cells using a transformation technique well-known to those skilled in the art. Type of the cells used for the transformation is not specifically limited.

The immunosuppressive dendritic cells collected as the result of the differentiation of the monocytes in the presence of the histatin-1 protein produce less pro-inflammatory cytokines such as IL-12 and produce more inhibitory cytokines such as IL-10 as compared with normal dendritic cells. In addition, they are capable of reducing the production amount of IFN-γ by T cells. Furthermore, the expression level of ULBP2 required for activating NK cells is reduced, and the expression level of CD1D required for activating NKT cells is also reduced. On the other hand, the expression level of TLR5, which is known to be expressed more on the immunosuppressive dendritic cells, is increased. Accordingly, the immunosuppressors may include immunosuppressive dendritic cells collected as the result of the differentiation of the monocytes by the action of the histatin-1 protein.

The immunosuppressive dendritic cells may appropriately be prepared from the monocytes by the action of the histatin-1 protein with an appropriate combination of, for example, a method of isolating monocytes that is well-known to those skilled in the art (e.g., Sumimoto H. et al., J. Exp. Med. 203: 1651-1656, 2006) and a culture method. An example is given below.

First, monocytes (hereinafter, also referred to as PBMCs) are isolated from the peripheral blood of a vertebrate and then CD14 positive monocytes are isolated. A method of isolating PBMCs is not specifically limited and can appropriately be determined by those skilled in the art depending on the type of PBMCs that are intended to be isolated. Ficoll centrifugation is an example of such a method. Moreover, CD14 positive monocytes can be isolated from the PBMC fractions using, for example, antibody-conjugated magnetic bead separation. The isolated CD14 positive monocytes are cultured in medium supplemented with serum including the histatin-1 protein, GM-CSF, and IL-4. The medium used may be, for example, RPMI1640 medium or AIM-V medium. Although not specifically limited as long as the immunosuppressive dendritic cells can be obtained, the culture period is preferably 5 to 6 days. Subsequently, the cells are cultured with stimulation by lipopolysaccharides and the histatin-1 protein and thus the dendritic cells can be obtained. In this culture process, the medium used may be, for example, RPMI1640 medium or AIM-V medium. In addition, although not specifically limited as long as the immunosuppressive dendritic cells can be obtained, the culture period is preferably 10 to 18 hours. In this case, the concentration of the histatin-1 protein is not specifically limited. The concentration is, however, preferably 5 μg/ml or higher, and more preferably, 10 μg/ml or higher. The upper limit is preferably 100 μg/ml or lower, more preferably, 50 μg/ml or lower, and yet more preferably 20 μg/ml or lower. The medium may appropriately be replaced at the discretion of those skilled in the art. For example, the medium may be replaced with half the volume of fresh medium every other day from the beginning of the culture.

Pharmaceutical additives well-known to those skilled in the art are used for formulation of the immunosuppressors, such as a pharmaceutically-acceptable carrier, diluent and vehicle. The dosage form is not specifically limited as long as it is suitable for delivering the present medicament from an injection site to a site where the immunosuppressor is intended to act, in the body of the animal to which the medicament is to be administered. For oral administration, the medicament may be formulated as oral drugs such as a tablet, a capsule, granules, powders, syrup, a enteric-coated tablet, a controlled-release capsule, a cachou, a chewable tablet, a drop, a pill, oral liquid, a confectionery tablet, a controlled-release tablet, and controlled-release granules. Alternatively, the medicament may be formulated as injectables for intracutaneous, subcutaneous, intravenous, intralymphatic, and intraperitoneal administrations. Thus, the injection site and the associated dosage form may appropriately be determined by a physician. The present medicament may be combined with a different pharmaceutical composition along with the aforementioned pharmaceutical additives.

### EXAMPLES

### [Melanoma Cell Lines]

Cell lines 397mel, 501mel, 526mel, 624mel, 888mel, 928mel, 938mel, 1102mel, A375, C32mel, G361mel, Malme3M, SKmel23, SKmel28, and WM266 used in the following Examples were obtained from Dr. Rousenberg, at the National Institutes of Health. A cell line MMG1 was obtained from Dr. Saita, at Shinshu University. Unless specifically noted, these cell lines were kept under 95% air and 5% CO₂ at 37° C. in RPM1640 supplemented with 10% FBS. Melanocytes were purchased from Kurabo and were incubated under 95% air and 5% CO₂ at 37° C. in 154S supplemented with Human Melanocyte Growth Supplement (HEMS).

### Example 1

This Example shows that histatin-1 is expressed in melanoma cell lines.

### ==Histatin-1 Gene Expression Analysis==

Total RNAs were extracted from each melanoma cell line using RNeasy total RNA isolation kit (Qiagen). From these RNAs, cDNAs were prepared using reverse Superscript III reverse-transcriptase and oligo (dT) primers (Invitrogen). These cDNAs were amplified via 35 cycles of PCR (denaturation: 94° C., 30 seconds; annealing: 57° C., 30 seconds; extension: 72° C., 60 seconds) using the following primers to analyze mRNA expression of histatin-1. GAPDH was used as the internal standard.
Histatin-1-F: cgctgattcacatgaaaagagac (SEQ ID No. 3)
Histatin-1-R: agggaagtatcatgaaacacaga (SEQ ID No. 4)

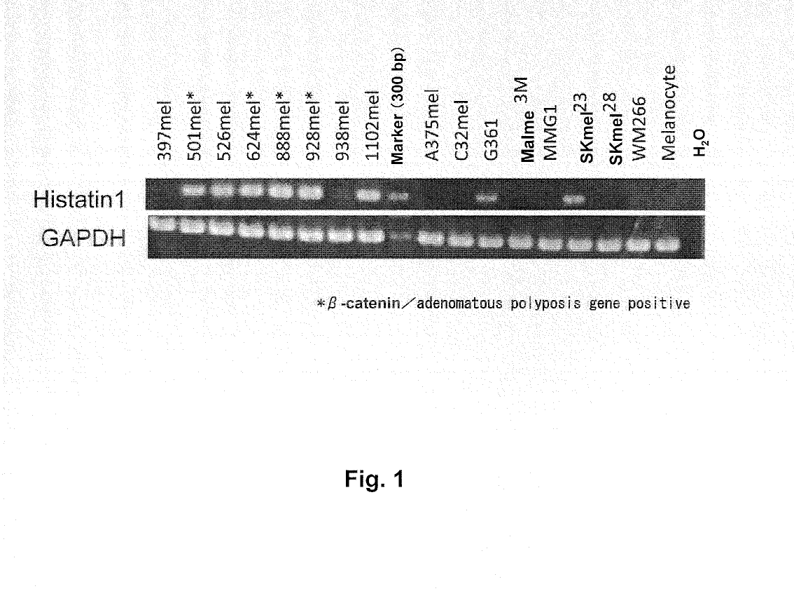

The result of the expression analysis is shown in FIG. 1. The histatin-1 mRNA was expressed in the eight cell lines (501mel, 526mel, 624mel, 888mel, 928mel, 1102mel, G361mel, and SKmel23) out of the sixteen human melanoma cell lines used for the analysis. On the other hand, no expression of the histatin-1 mRNA was detected in normal melanocytes. This result indicates that histatin-1 can be used as a diagnostic marker for melanoma.

### Example 2

This Example shows that the expression of the histatin-1 mRNA in tumor tissues is correlated to life expectancy.

Metastatic lesions in the sentinel lymph nodes were collected from 25 patients with melanoma at Shinshu University School of Medicine. RNAs were isolated from the collected tissue and quantitative PCR was performed using a TaqMan RNA-to-CT 1-Step kit to analyze the expression of the histatin-1 mRNAs in the tissue of each patient.

A group of 13 patients with high expression of histatin-1 mRNA and a group of 12 patients with low expression of histatin-1 mRNA were periodically followed up for their survival rate for 90 months after the expression analysis of the histatin-1 mRNA. The expression level of the histatin-1 mRNA was normalized to that of GAPDH. The patients were divided into two groups according to whether the expression level is higher than −6.25 (high-expression group) or equal to or lower than −6.25 (low-expression group), in which the value −6.25 is the mean of the expression levels after log-transformation.

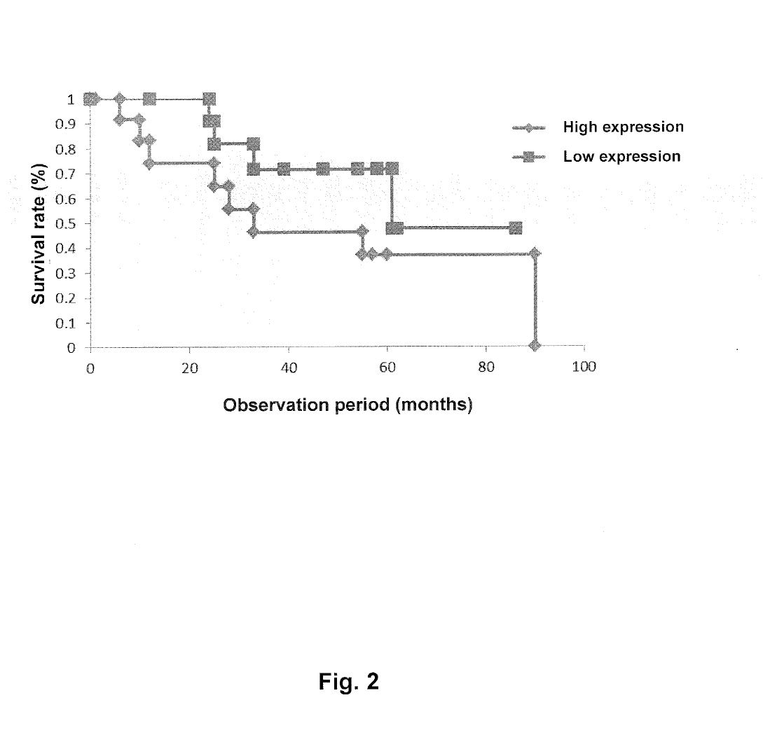

As shown in FIG. 2, the survival rate of the patients in the high-expression group is always higher than that of the patients in the low-expression group during the observation. This result indicates that the expression level of the histatin-1 mRNA in tumor cells of melanoma correlates to the life expectancy. Accordingly, the histatin-1 mRNA and the histatin-1 protein can be used as the marker for determining the prognosis or life expectancy of patients having melanoma.

### Example 3

This Example indicates that the expression of the histatin-1 protein and the histatin-1 gene enhances the migration capacity of melanoma cells and that the knock-down of the histatin-1 gene reduces the migration capacity of the melanoma cells.

### ==Addition of Histatin-1 Protein==

The C32mel was seeded into wells in the upper chamber of a 16-well plate for cell migration assay (CIM-plate 16, Roche Diagnostics GmbH) at a density of 3×10⁴ cells/well. The wells in the upper chamber were filled with serum-free RPMI1640 while the wells in the lower chamber were filled with RPMI1640 supplemented with 10% FBS. Then, 0, 5, 10 or 20 μg/ml of histatin-1 protein was added to each well in the upper chamber and was subjected to the analysis of the migration capacity.

### ==Knock-Down-of Histatin-1 Gene==

50 μM of siRNA targeted to the following two sequences (SEQ ID Nos. 5 and 6) in the histatin-1 mRNA was transfected to SKmel23 (4×10⁴ cells/500 μl/well (24-well plate)) using LipofectaMINE2000 (Invitrogen) 1 μ/well. Stealth RNAi Negative Control Low GC Duplex (Invitrogen) was used as a negative control siRNA.

(SEQ ID No. 5)

| | |
|---|---|
| siRNA-target-seq2: | UAUAGAUAAUUUGAUCCAUAGUCCC |

(SEQ ID No. 6)

| | |
|---|---|
| siRNA-target-seq3: | AAAUCAUGAGAGCCAAGACUAAAGC |

Transfection efficiency was determined using BLOCK-iT Fluorescent Oligo (Invitrogen) and found to be 90% or higher.

The cell line SKmel23 into which siRNA has been transfected was seeded into wells in the upper chamber of CIM-plate 16 at a density of 1×10⁵ cells/well for the following migration assays.

### ==Forced Expression of Histatin-1 Gene==

Histatin-1 cDNA was cloned into the multiple cloning site of pCSII-CMV-MCS-IRES (internal ribosomal site)-puroR-PRE vector (obtained from Dr. Miyoshi at RIKEN; Miyoshi H. et al., J. Virol. 72(10): 8150-8157, 1998), which was prepared by inserting a puromycin resistance cassette IRES-puroR into a pCSTI-CMV-MCS-PRE vector. The vector with the histatin-1 cDNA was co-transfected with pCAG-HIVgp (3rd generation packaging plasmid) and pCMV-VSV-G-RSV-Rev (plasmid expressing VSV-G envelope protein and Rev protein) into HEK293 cells to prepare an HIV-derived third generation virus expressing histatin-1 (hereinafter, also referred to as a histatin-1 expressing virus). The supernatant was collected and concentrated as a stock of the histatin-1 expressing virus.

A375 seeded at a density of 5×10⁴ cells/2 ml/well on 6-well plates was infected with the aforementioned histatin-1 expressing virus added at a density of 15 to 30 μl/well. Four days after the infection. A375 was selected with puromycin. In this way, A375 in which the histatin-1 gene was forcibly expressed was selected.

This A375 cell was seeded into wells in the upper chamber of CIM-plate 16 at a density of 3×10⁴ cells/well and the cell migration assay was performed.

### ==Melanoma Cell Migration Assay==

The wells in the upper chamber of the CIM-plate were filled with serum-free RPMI1640, while the wells in the lower chamber were filled with RPMI1640 supplemented with 10% FBS. The plate was placed in RTCA DP Instrument of the xCELLigence system (Roche Diagnostics GmbH) and cell migration was monitored every 15 minutes under 95% air and 5% CO, at 37° C.

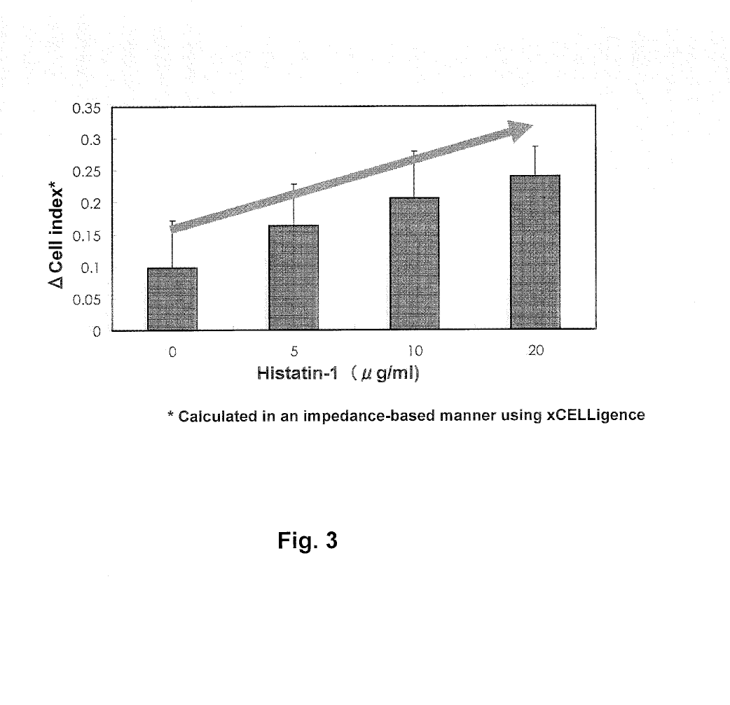

As shown in FIG. 3, the migration capacity of the tumor cells was enhanced in the case where the histatin-1 protein was added to the melanoma cells.

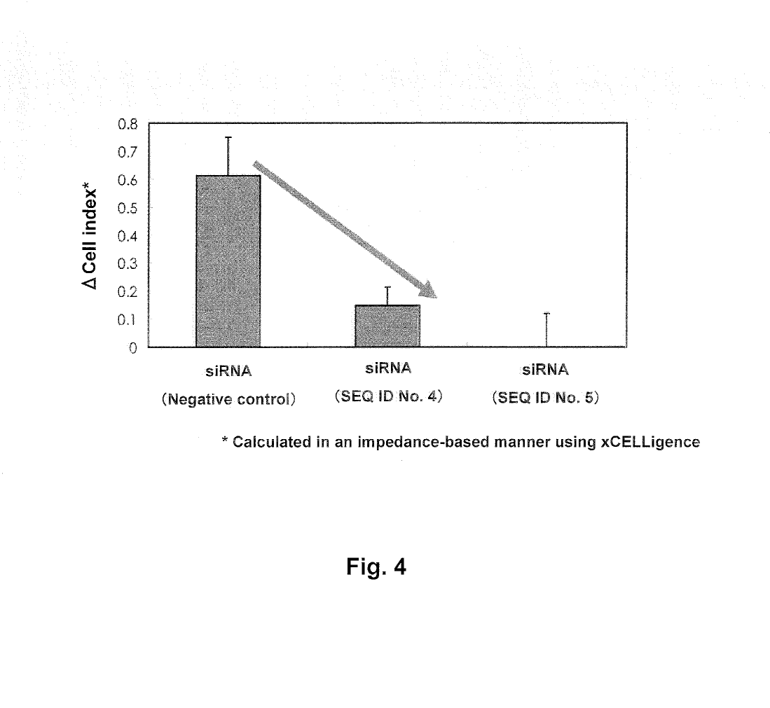

As shown in FIG. 4, the migration capacity of the tumor cells was reduced in the case where the histatin-1 gene of the melanoma cells was knocked down.

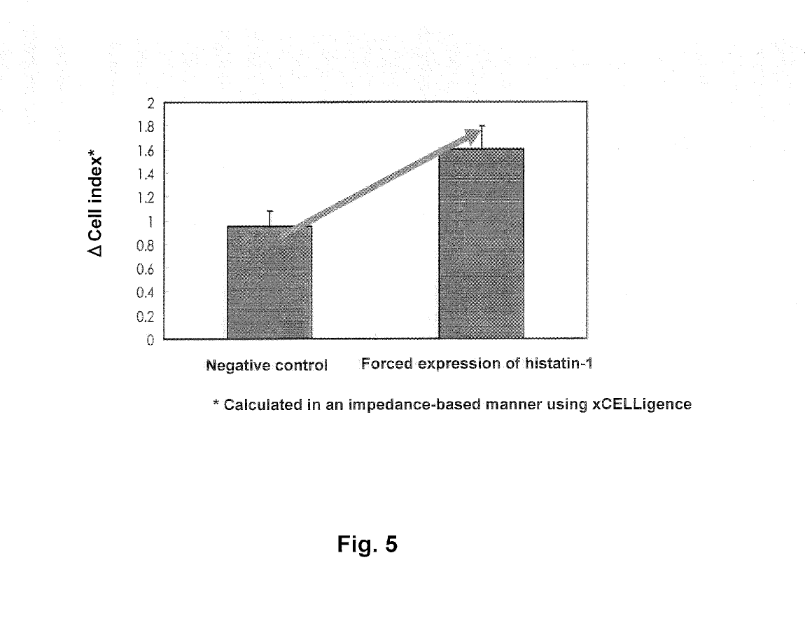

As shown in FIG. 5, the forced expression of the histatin-1 gene in the melanoma cells enhanced the migration capacity of the tumor cells.

These results indicate that the histatin-1 protein is associated with the migration capacity of the tumor cells, and that the migration capacity, of the melanoma cells can be reduced by suppressing the function of the histatin-1 protein.

### EXAMPLE 4

This Example shows that the invasive potential and the metastatic potential are enhanced in melanoma cells in which the histatin-1 gene is forcibly expressed.

According to the description of the “Forced expression of Histatin-1 gene” in Example3, A375 was infected with the histatin-1 expressing virus. Then, A375 in which histatin-1 was forcibly expressed was selected with puromycin.

Furthermore, in the description of the “Forced expression of Histatin-1 gene” in Example3, cDNA of a modified GFP gene rather than the histatin-1 cDNA was cloned into the multiple cloning site of pCSII-CMV-MCS-PRE vector to prepare an HIV-derived third generation GFP expressing virus. The supernatant was collected and concentrated as a stock of the GFP expressing virus. The cell line A375 expressing the histatin-1 gene selected with puromycin was seeded at a density of 5×10⁴ cells/2 ml/well on 6-well plates and infected with the GFP expressing virus added at a density of 15 to 30 μl/well. Four days after the infection, the expression of GFP was observed under fluorescence microscope (GFP expression of almost 100% was confirmed). A375 expressing histatin-1/GFP was thus obtained.

Subsequently, 3 to 5×10⁶ cells/animal of A375 expressing histatin-l/GFP was implanted into the flank of nude mice (BALB/c nu/nu; five-week old, female). The mice were bred for 2 to 3 weeks (N=5). Axillary lymph nodes and inguinal lymph nodes, which are lymph nodes on the same side as the implantation site, and lung were removed. The axillary and inguinal lymph nodes were minced with scissors in a serum-free RPMI1640 medium to dissociate the cells. Lung tissues were minced with scissors in a serum-free RPMI1640 medium and digested with collagenase to dissociate the cells. Normal mice that were not subjected to cell implantation were used as NO TUMOR CELLS group (N=5). An MOCK group (N=4) consists of the mice that received the implantation of A375 expressing no histatin-1 and expressing GFP, infected with a virus having no histatin-1 cDNA.

The ratio of GFP positive cells to the dissociated cells was determined using FACS.

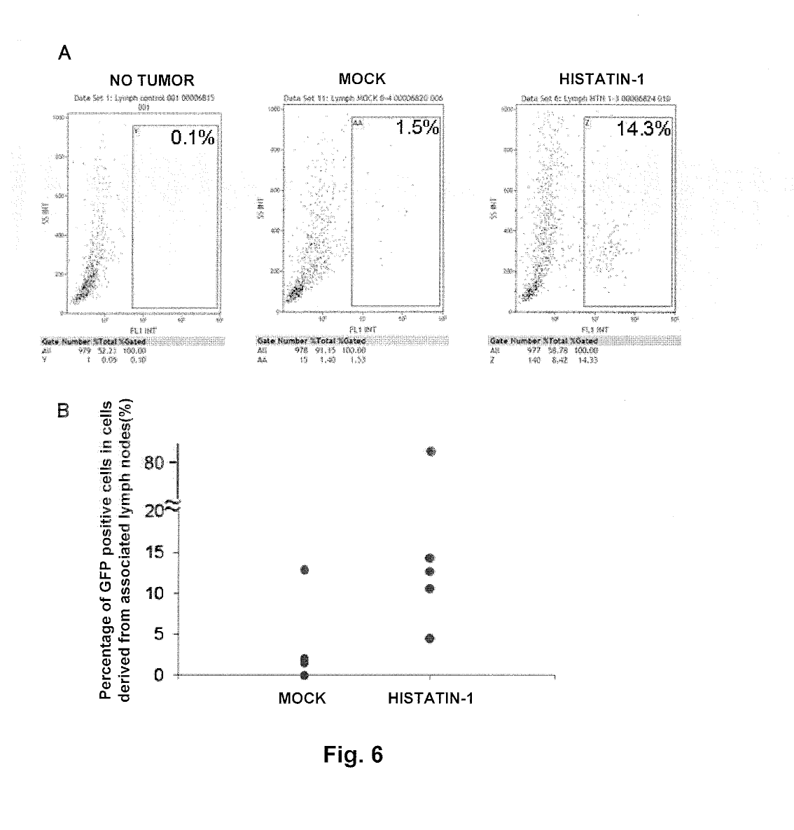

FIGS. 6A and 6B show rates of GFP positive cells in the cells collected from the associated lymph nodes (axillary and inguinal lymph nodes). The group with A375 expressing histatin-1/GFP implanted (HISTATIN-1) exhibited a significantly higher rate of GFP positive cells in the cells collected from the associated lymph nodes as compared with the MOCK group.

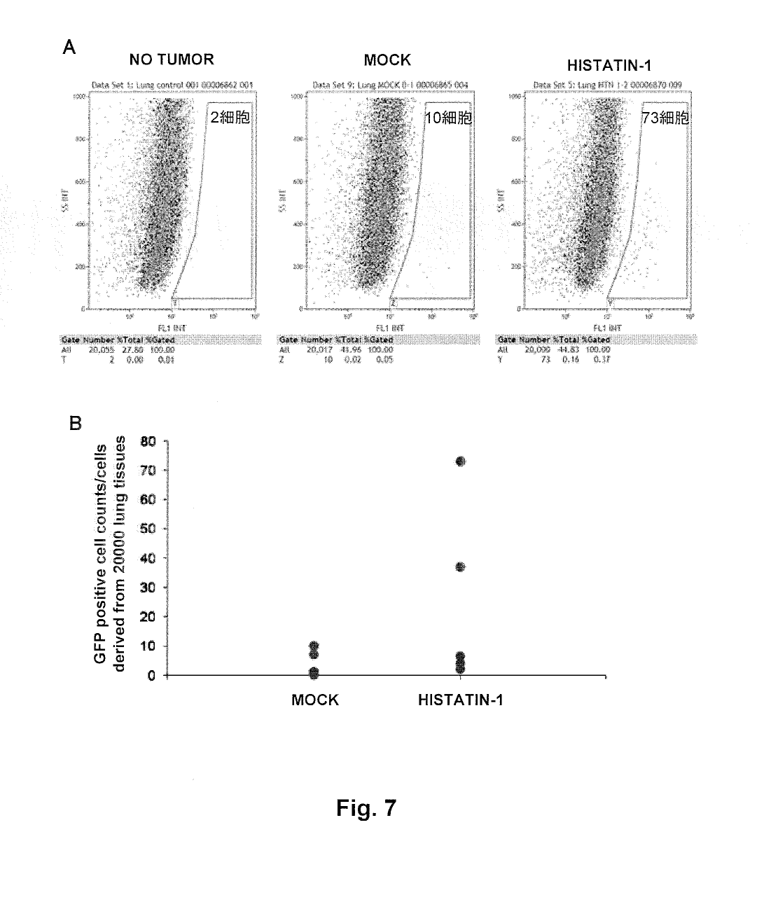

FIGS. 7A and 7B show the count of GFP positive cells out of 20,000 cells collected from lung tissues. The group with A375 expressing histatin-1/CFP implanted (HISTATIN-1) exhibited a significantly higher rate of GFP positive cells in the cells collected from the lung as compared with the MOCK group.

Since the GFP positive cells were implanted into the flank, the aforementioned result indicates that invasion and metastasis to the associated lymph nodes and the lung are enhanced with A375 expressing histatin-1 as compared to A375 not expressing histatin-1.

This means that the histatin-1 of melanoma can be used as diagnostic markers with which the invasive potential or the metastatic potential of melanoma can be determined. In addition, they can be used as a therapeutic agent against melanoma that suppresses invasion and metastasis of melanoma by inhibiting the function or the expression of histatin-1.

### EXAMPLE 5

This Example shows that immunosuppressive dendritic cells can be prepared using the histatin-1 protein.

### ==Preparation of Dendritic Cells==

Blood was obtained from a healthy subject who gave informed consent at School of Medicine, Keio University. To about 100 ml of this blood, approximately 1 ml of heparin was added. The mixture was overlaid on Lymphoprep (Axis-Shield PoC, AS) and centrifuged (2000 rpm, 30 minutes, room temperature). The intermediate layer containing PBMCs was separated as a PBMC fraction. CD14 positive monocytes were isolated from the PBMCs using CD14 Micro-Beads (Miltenyi Biotec). The CD14 positive monocytes were plated with an RPMI1640 medium supplemented with 10% FCS at a density of 2 to 2.5×10⁴ cells/well/2 ml, to which GM-CSF (100 ng/ml), IL-4 (50 ng/ml), and the histatin-1 protein (0, 5 or 10 μg/ml) were added before incubation. The cells were incubated for 6 days with the medium being replaced with half the volume of fresh medium every other day to differentiate into dendritic cells. These dendritic cells were washed and plated again at a density of 1 to 2.0×10⁴ cells/2 ml/well, to which the histatin-1 protein (0, 5 or 10 μg/ml) and liposaccharide (1 μg/ml) were added. Incubation was continued for additional 10 to 18 hours. It is noted that the entire incubation was performed under the condition of 5% Co₂ at 37° C.

The medium was recovered after the completion of the incubation, and IL-10, TNF-α, and IL-12 produced by the dendritic cells and secreted into the medium were measured using an ELISA kit (BD Biosciences Pharmingen). In addition, for the dendritic cells thus obtained, expression of cell surface markers CD14, CD80, CD83, CD86, and HLA-DR was measured using FACS.

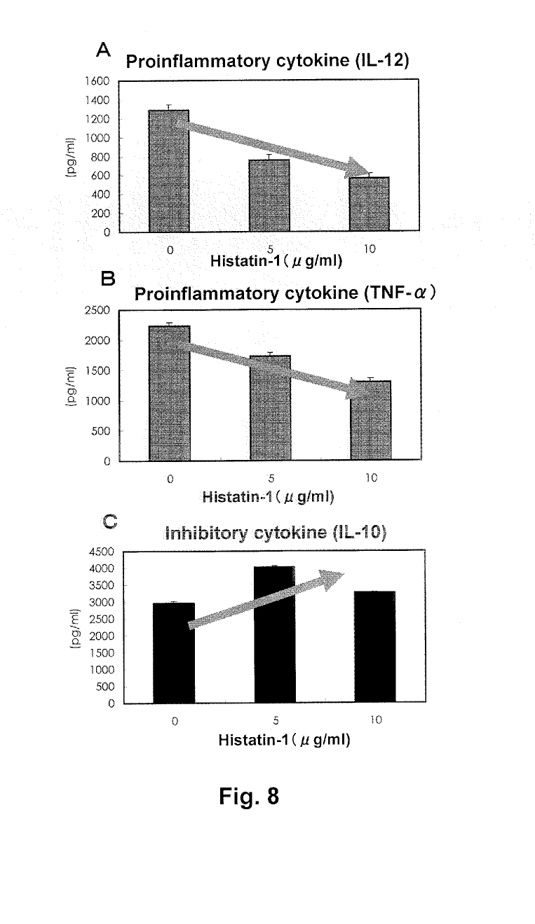

As shown in FIG. 8, the amount of the proinflammatory cytokines IL-12 and TNF-α was significantly decreased depending on the concentration of the histatin-1 protein added (A, B). On the other hand, the amount of the inhibitory cytokine IL-10 was significantly increased when the concentration of the histatin-1 protein is 5 μg/ml. The amount was also increased when the concentration was 10 μg/ml, as compared to the case of 0 μg/ml (C).

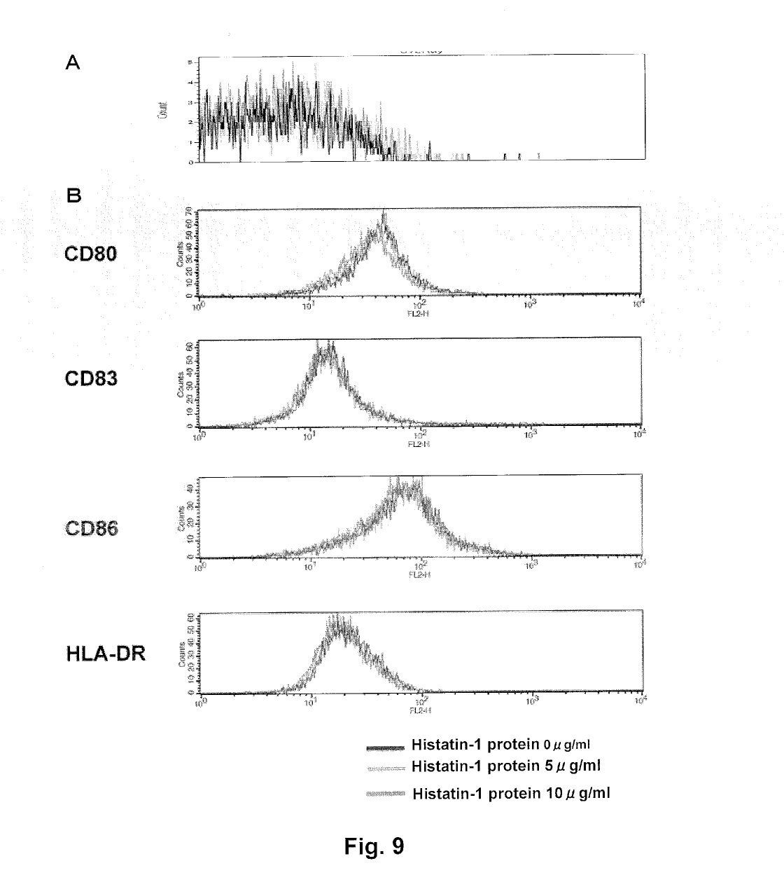

As shown in FIG. 9, the dendritic cells obtained were all CD14 negative (FIG. 9A), and expression level of each of CD80, CD83, CD86 and HLA-DR was constant (FIG. 9E) regardless of the concentration of the histatin-1 protein in both cases where the dendritic cells were differentiated without the addition of the histatin-1 protein and where the dendritic cells were differentiated with the addition of the histatin-1 protein. This result indicates that the dendritic cells differentiated with the addition of the histatin-1 protein have expression of the markers similar to those provided with the dendritic cells differentiated in a conventional manner without the addition of the histatin-1 protein, and that the expression of the markers is not dependent of the concentration of the histatin-1 protein. In addition, regardless of the amount of the histatin-1 protein added the expressions of the respective cell surface markers have a single peak and a similar shape (FIG. 9B). This indicates that histatin-1 does not inhibit the differentiation of the monocytes into the dendritic cells; instead, a population of the dendritic cells obtained with the addition of the histatin-1 protein is homogeneous at the same degree as in a population of the dendritic cells obtained without the addition of the histatin-1 protein.

Thus, regardless the amount of the histatin-1 protein added, the population of the dendritic cells obtained from the CD14 positive monocytes with the addition of the histatin-1 protein has the expression of the markers that are identical to those expressed in the population of the dendritic cells differentiated without the addition of the histatin-1 protein. The former population is, however, different from the latter in that the cells in the former population have an inhibitory function.

### ==Stimulation of T Cells by Dendritic Cells==

T cells were isolated from the blood collected from the aforementioned healthy subject using CD3 MACS beads (Miltenyi Biotec). These T cells were seeded on a 96-well plate at a density of 2.0×10⁵ cells along with the dendritic cells (DCs, irradiated with a total dose of 32 Gy for about 20 minutes) prepared in the manner described above, at a density of 2.0 (DCs:T cells=1:10), 0.67 (DCs:T cells=1:30) or 0.22×10⁴ cells (DCs:T cells=1:90). The cells were incubated under 95% air and 5% CO₂ at 37° C. in 200 μl/well of AIM-V medium supplemented with 5% human AB serum. Three days after the initiation of the incubation, the medium was recovered and IFN-γ produced from the T cells was measured using the ELISA kit (M700A, M701B, both from Endogen).

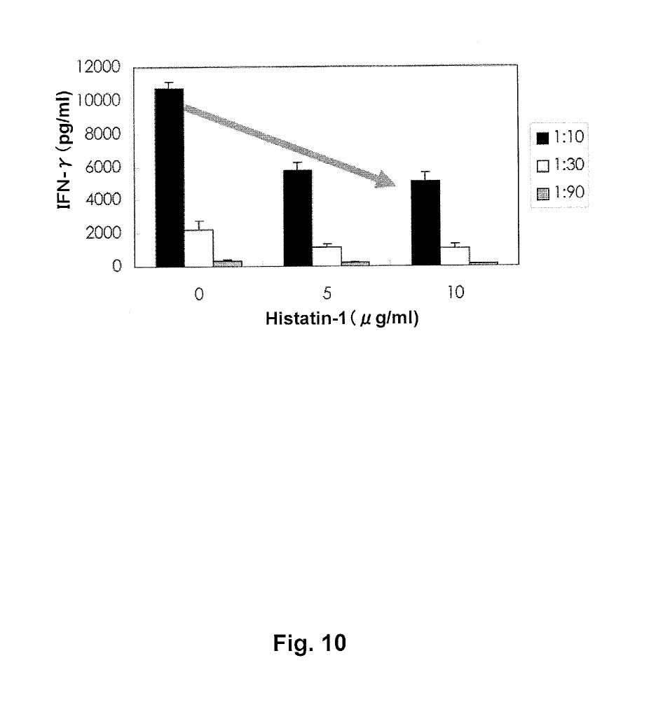

As shown in FIG. 10, when the ratio of dendritic cells:T cells is 1:10, the amount of IFN-γ was decreased dependent of the concentration of the histatin-1 protein used in the differentiation process of the dendritic cells. T cells produce IFN-γ in response to stimulation with dendritic cells. Thus, this result indicates that the ability of the dendritic cells differentiated from monocytes under the action of the histatin-1 protein to stimulate T cells decreases dependent on the concentration of the histatin-1 protein.

Thus, the histatin-1 protein acts on monocytes and serves to direct the differentiation of dendritic cells to be immunosuppressive. The dendritic cells differentiated under the action of the histatin-1 protein produce less proinflammatory cytokines and more immunosuppressive cytokines, and have a decreased ability to stimulate T cells. Accordingly, the histatin-1 protein and the dendritic cells differentiated using the histatin-1 protein both have an immunosuppressive function.

### ==Genes Expressed in Dendritic Cells==

Total RNAs were extracted from the dendritic cells differentiated without the addition of the histatin-1 protein (normally-differentiated dendritic cells) that were obtained as described in the “Preparation of dendritic cells” and the dendritic cells differentiated with the addition of the histatin-1 protein (HTN dendritic cells) using a RNeasy total RNA isolation kit (Qiagen). These RNAs were used to globally analyze gene expression with Gene Chip (Aglient; Whole Human Genome oligo DNA microarray kit (4×44K)).

As shown in Table 1 below, expression level of 5,458 genes was enhanced 1.5-fold or more in the dendritic cells differentiated with the addition of the histatin-1 protein (HTN dendritic cells) as compared with the dendritic cells differentiated without the addition of the histatin-1 protein (normally-differentiated dendritic cells). In addition, in the HTN dendritic cells, the expression level of ULBP2 required to activate NK cells was decreased to 1/20 while the expression level of CD1d required to activate NKT cells was decreased to 3/10 as compared with the normally-differentiated dendritic cells. On the other hand, the expression level of TLR5 (Vicente-Suarez I et al., Immunol Lett. 125 114-8, 2009) known to be largely expressed in immunosuppressive dendritic cells was 48.4 times higher in the HTN dendritic cells than in the normally-differentiated dendritic cells. Furthermore, the expression level of CCR4, which are typically known to be expressed preferentially in regulatory T cells, was 108.26 times higher in the HTN dendritic cells than in the normally-differentiated dendritic cells.

TABLE 1

| | | Normally- | |
|---|---|---|---|
| | | differentiated | |
| | | DCs | HTN DCs |
| TLR5 | Signal | 1.64 | 79.30 |
| | Normalized | 1 | 48.42 |
| CCR4 | Signal | 1.64 | 177.65 |
| | Normalized | 1 | 108.26 |
| ULBP2 | Signal | 119.53 | 6.35 |
| | Normalized | 1 | 0.053 |
| CD1d | Signal | 12.45 | 3.81 |
| | Normalized | 1 | 0.305 |

Thus, a significant difference exists in gene expression between the dendritic cells (HTN dendritic cells) obtained from the CD14 positive monocytes with the addition of the histatin-1 protein and the normally-differentiated dendritic cells. In particular, the gene expression profile of the dendritic cells obtained with the addition of the histatin-1 protein indicates that these cells have an immunosuppressive function.

### Example 6

This Example shows that characteristics of dendritic cells can be altered in vivo in such a manner that they have an inhibitory function by administering cells in which the histatin-1 protein is forcibly expressed to a mouse.

Human melanoma 397mel plated at a density of 5×10⁴ cells/2 ml/well (6-well plate) was infected with the virus expressing histatin-1 described in Example 3 added at a density of 15 to 30 μl/well. Four days after the infection, 397mel was selected with puromycin. In this way, clones in which the histatin-1 gene was forcibly expressed were selected as 397mel-Histatin-1.

This 397mel-Histatin-1 was suspended in 100 μl of RPMI1640, which was subcutaneously inoculated to a lower leg of nude mice (BALE/c nu/nu; five-week old; female) at a density of 3×10⁶ cells/animal. As a negative control group, mice were inoculated with clones (397mel-MOCK) obtained by infecting 397mel with an HIV virus without histatin-1. About 30 days later, tumor tissues derived from the inoculated 397mel and the spleen were isolated from the mice in both groups. CD11c positive dendritic cells were isolated from each of them using CD11c MACS beads (Miltenyi Biotec). These dendritic cells were assayed for their ability to activate T cells in the following manner.

First, T cells were isolated from the spleen of a mouse (Balb/c) using CD90.2 microbeads (Miltenyi Biotec). On the other hand, the isolated dendritic cells were irradiated with a total dose of 32 Gy for about 20 minutes to arrest cell division. Then, T cells and dendritic cells were plated at densities 1.6×10⁵ and 1.6×10⁴, respectively, with 200 μl/well (96-well plate) of culture medium (RPMI1640 supplemented with 10% FBS). Anti-mouse CD3 antibody (1 μg/ml) (BD Biosciences Pharmingen) was added to the medium and the cells were incubated in the presence of 5% CO₂ at 37° C. Four days after the initiation of the incubation, the medium was recovered and IFN-γ produced from the T cells was measured using the ELISA kit (BD Biosciences Pharmingen).

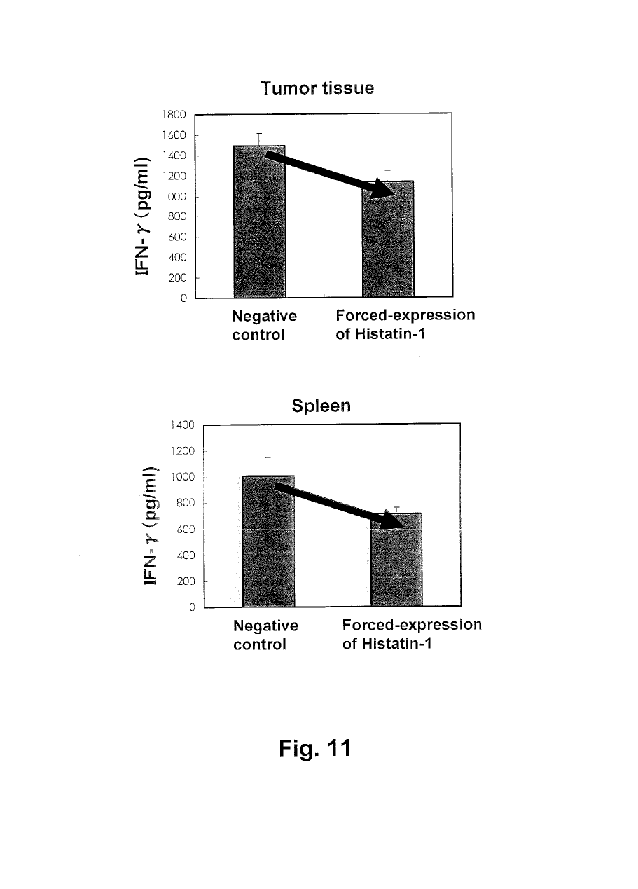

As shown in FIG. 11, in both of the tumor tissues and the spleen, the dendritic cells derived from the mice that had received subcutaneous inoculation of 397mel-Histatin-1 had lower ability to activate the T cells than the dendritic cells derived from the mice that had received subcutaneous inoculation of 397mel-MOCK as measured by the amount of the IFN-γ produced from the T cells. This indicates that the exogenous histatin-1 protein expressed in 397mel suppressed the function of the dendritic cells in vivo. In addition, the histatin-1 protein suppressed the function of not only the dendritic cells present in the tumor tissues derived from 397mel but also the dendritic cells derived from the spleen. This indicates that the histatin-1 protein secreted from 397mel acts systemically via the blood.

### INDUSTRIAL APPLICABILITY

The present invention provides diagnostic markers for melanoma.

The present invention also provides novel pharmaceutical compositions.

Furthermore, the present invention provides novel immunosuppressors.

1. An immunosuppressor comprising a histatin-1 protein.

2. The immunosuppressor according to claim 1, comprising an expression vector capable of expressing the histatin-1 protein.

3. The immunosuppressor according to claim 2, comprising a transformant transformed with the expression vector.

4. A method of producing an immunosuppressive dendritic cell comprising the step of:
differentiating a monocyte isolated from human or a non-human vertebrate in the presence of a histatin-1 protein.
